(19) United States
(12) Patent Application Publication
(10) Pub. No.: US 2005/0267507 A1
(43) Pub. Date: Dec. 1, 2005

(54) Apparatus for acupuncture needle set

(76) Inventor: Beaver Kao, Zhonghe City (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 10/845,043

(22) Filed: May 13, 2004

(51) Int. Cl. A61B 17/34

(52) U.S. Cl. 606/189

(57) ABSTRACT

The present invention generally relates to an apparatus for an improved acupuncture needle set. The improved acupuncture needle set at least comprises an acupuncture needle and a needle case. In accordance with the design of this present invention, the acupuncture needle can be fixed in the needle case by the fastening structures inside the needle case and at the acupuncture needle. Moreover, appropriate pressure is punched on the acupuncture needle by an acupuncturist to release the needle by overcoming the resistant friction from the fastening structures. Hence, this invention provides an improved acupuncture needle set, which could be manipulated in one hand. It also improves the convenience and accuracy of the insertion process of acupuncture.

### BACKGROUND OF THE INVENTION

1. Field of the Invention

The present invention generally relates to needles, and more particularly to an apparatus for acupuncture needle set.

2. Description of the Prior Art

Traditional Chinese Medicine (TCM) plays a quite important role in modern medical development. For many western researchers, medical theories and treatments in TCM are very attractive and mystical. Acupuncture is one of these theories and treatments. Acupuncture in TCM is a special medical treatment process, that the acupuncturists use needles to stimulate some acupuncture points (or acupoints in abbreviation) of the patient body.

In early times, most acupuncturists just use their fingers to insert the needles into the acupoints of the patient body in the acupuncture treatment process. Today, for the convenience of the acupuncturists in the clinical treatment process, they manipulate the acupuncture needle set as illustrated in FIG. 1. The acupuncture needle set shown in FIG. 1 at least comprises an acupuncture needle 120 and a needle case 140. The main function of the needle case 140 is used to make the insertion of the needle more conveniently and accurately.

In the prior art, referring to FIG. 1, the acupuncture needle 120 could be placed inside the needle case 140, and a needle fastening element 160 is used to fix the acupuncture needle 120 in the needle case 140 in order to prevent the acupuncture needle 120 dropping out of the needle case 140. Before the acupuncturist makes the insertion, the acupuncture set is aimed at an acupoint and the needle case 140 is placed at the skin. The needle case 140 is hold by one hand and the needle fastening element 160 is unfastened by the other hand to release the acupuncture needle 120. Then the acupuncture needle 120 is inserted into a proper depth. The mentioned acupuncture needle set could help the acupuncturist accurately and conveniently to locate the insertion point and the probability to insert into wrong point could be also efficiently reduced by the mentioned design. However, both hands of the acupuncturist are required to manipulate the mentioned acupuncture needle set at the same time. The acupuncturist is unable to use his hands at will to do others during the insertion process. The most serious case is happened at the moments when the needle fastening element 160 is released by one of the acupuncturist's hands; the other hand that holds the needle case 140 for pinpointing the acupuncture needle 120 to the exact acupoint may unintentionally move or drift slightly. The unintentional error of insertion point may reduce the medical effects or even cause serious undesired side effects.

Accordingly, it is desired to provide an improved acupuncture needle set to improve the accuracy of acupuncture and the manipulation of acupuncture process.

### SUMMARY OF THE INVENTION

In accordance with the present invention, an acupuncture needle set is provided. The acupuncture needle set of this present invention employs a fastening structure to fix an acupuncture needle to a needle case. The improved acupuncture needle set could be handled conveniently by an acupuncturist. Further, the improved acupuncture needle set could be manipulated by one hand. The acupuncture insertion process is also improved and the error probability in the acupuncture insertion process could be reduced.

To achieve certain advantages and novel features, the present invention is generally directed to an improved acupuncture needle set. The presented acupuncture needle set at least comprises an acupuncture needle and a needle case. The mentioned needle case further comprises a first fastening structure located at the proper position inside the needle case. Moreover, the mentioned acupuncture needle comprises a needle body and a needle holder. The acupuncture needle set further comprises a second fastening structure located at the needle holder. In accordance of this present invention, the first fastening structure and the second fastening structure are utilized to fix the acupuncture needle at a proper position inside the needle case. Hence, the design of this invention does not only make the insertion manipulation more conveniently but also reduce the probable error caused by using both hands of the acupuncturist.

In one particular embodiment, the present invention provides an improved acupuncture needle set, which could be easily manipulated in single hand. In accordance of this embodiment, the needle case is pinpointed at the acupoint in one hand. Further, the acupuncturist could use one finger of the same hand to press on the exposed end of the acupuncture needle to overcome the resistant friction between the first fastening structure and the second fastening structure. Then, the acupuncture needle is released to breakthrough the skin of the patient. In other words, the design of this present invention provides an improved acupuncture needle set that could be manipulated in single hand and makes the acupuncture process more conveniently and accurately for the acupuncturist.

### BRIEF DESCRIPTION OF THE DRAWINGS

The accompanying drawings incorporated in and forming a part of the specification illustrate several aspects of the present invention, and together with the description serve to explain the principles of the invention. In the drawings:

FIG. 1 shows a diagram illustrating an acupuncture needle set in the prior art;

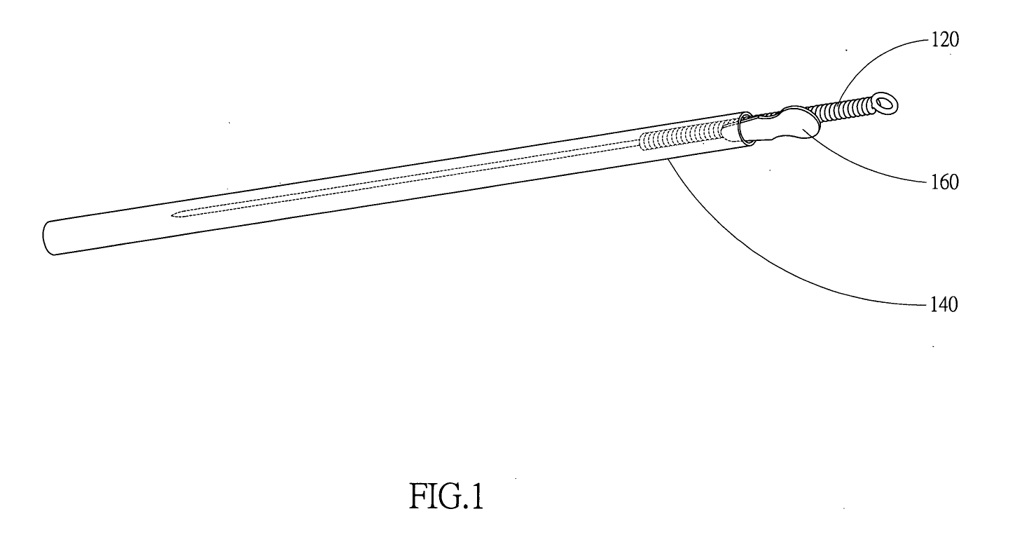

FIG. 2 depicts a diagram illustrating compositions of an improved acupuncture needle set, constructed in accordance with an embodiment of the invention;

FIG. 3 shows a diagram illustrating a fastening structure of an improved acupuncture needle set, constructed in accordance with an embodiment of the invention;

FIG. 4 shows a diagram illustrating compositions of an improved acupuncture needle set, constructed in accordance with an embodiment of the invention;

FIG. 5 shows a diagram illustrating compositions of an improved acupuncture needle set, constructed in accordance with an embodiment of the invention; and

FIG. 6 shows a diagram illustrating compositions of an improved acupuncture needle set, constructed in accordance with an embodiment of the invention.

### DESCRIPTION OF THE PREFERRED EMBODIMENT(S)

Having summarized various aspects of the present invention, reference will now be made in detail to the description of the invention as illustrated in the drawings. While the invention will be described in connection with these drawings, there is no intent to limit it to the embodiment or embodiments disclosed therein. On the contrary the intent is to cover all alternatives, modifications and equivalents included within the spirit and scope of the invention as defined by the appended claims.

It is noted that the drawings presents herein have been provided to illustrate certain features and aspects of embodiments of the invention. It will be appreciated from the description provided herein that a variety of alternative embodiments and implementations may be realized, consistent with the scope and spirit of the present invention.

It is also noted that the drawings presents herein are not consistent with the same scale. Some scales of some components are not proportional to the scales of other components in order to provide comprehensive descriptions and emphasizes to this present invention.

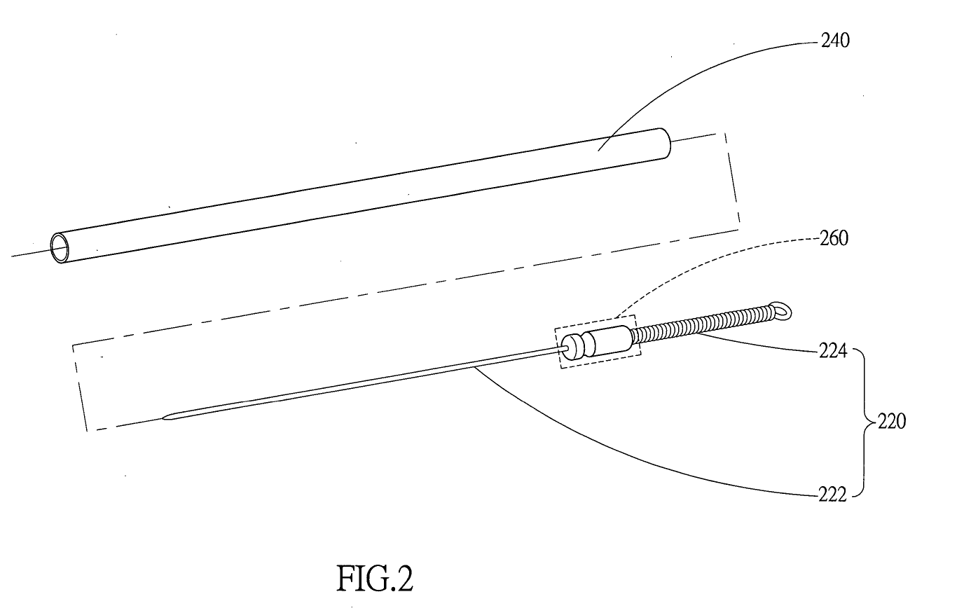

One of preferred embodiments of the present invention is an improved acupuncture needle set. FIG. 2 is a diagram depicts the improved acupuncture needle set in accordance with this embodiment. Referring to FIG. 2, the mentioned acupuncture needle set at least comprises an acupuncture needle 220 and a needle case 240. The needle case 240 could further comprise a first fastening structure 242. The first fastening structure 242 is located at a proper position inside the needle case 240. The first fastening structure 242 may be a circle of internal bulge or at least a projecting point inside the needle case 240. Other well-known fastening structures are also acceptable for the first fastening structure 242.

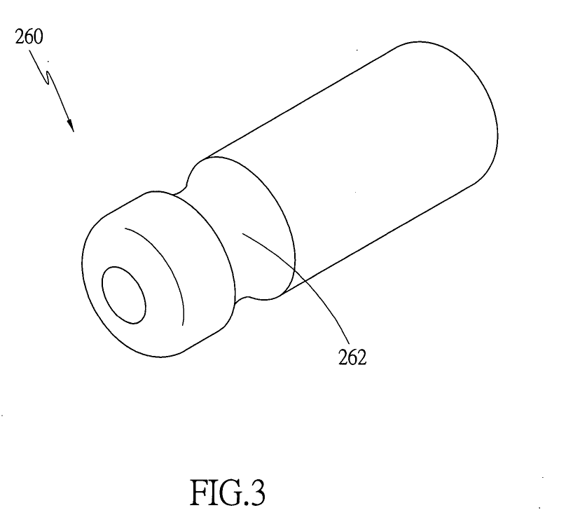

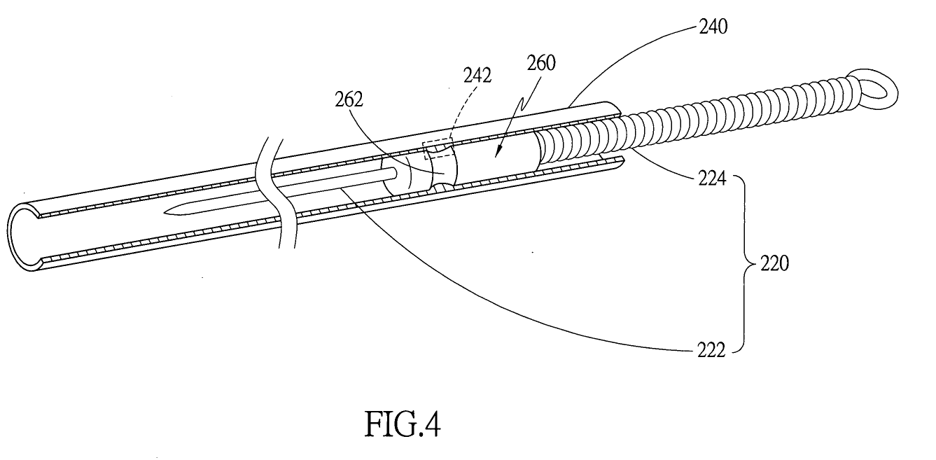

The acupuncture needle 220 comprises a needle body 222 and a needle holder 224. The needle holder can be twisted by fingers to help inserting the acupuncture needle 220 into an acupoint. Both of the needle body 222 and the needle holder 224 may be made of metal. The improved acupuncture needle set may further comprise a second fastening structure 260 positioned at the needle holder 224. Furthermore, the second fastening structure 260 may comprise a flute 262, as shown in FIG. 3. The second fastening structure 260 could be made of plastic or other materials. Referring to FIG. 4, the flute 262 of the second fastening structure 260 could be matched with the bulge of the first fastening structure 242. The matched joint of these two fastening structure 242 and 260 could fix the acupuncture needle 220 at a proper position inside the needle case 240. Accordance with one preferred example of this embodiment, the first fastening structure 260 could be placed nearby the lower end of the needle holder 224.

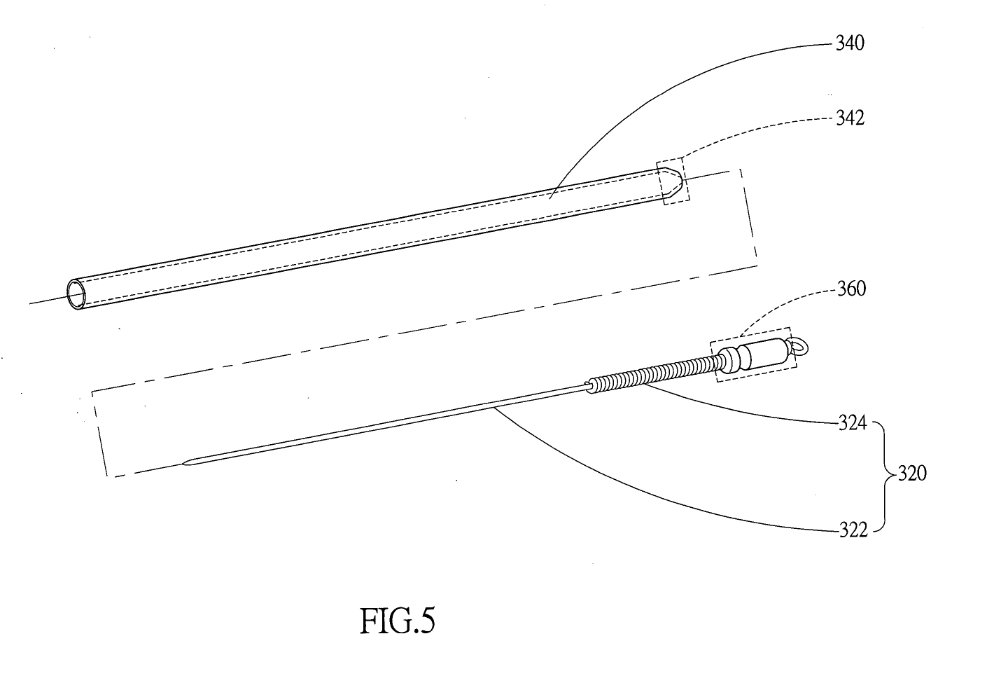

Another preferred embodiment of the present invention is an improved acupuncture needle set. Referring to FIG. 5, the illustrated acupuncture needle set at least comprises an acupuncture needle 320 and a needle case 340. The mentioned needle case 340 may further comprise a first fastening structure 342. The first fastening structure is located at one opening of the needle case 340, and the caliber of the mentioned opening is smaller than the caliber of another opening of the needle case 340.

Accordance with this embodiment, the acupuncture needle 320 comprises a needle body 322 and a needle holder 324. Both of the needle body 322 and the needle body 324 could be made of metal. The improved acupuncture needle set may further comprise a second fastening structure 360. The second fastening structure 360 may be made of plastic or other materials. The second fastening structure 360 mentioned above could be placed at the needle holder 324. Furthermore, the second fastening structure 360 may comprises a flute 362. Accordance with this embodiment, the second fastening structure 360 could be placed nearby the upper end of the needle holder 324, as is shown in FIG. 5. The flute 362 of the second fastening structure 360 could be matched with the bulge of the first fastening structure 342. The matched joint of these two fastening structures 342 and 360 could fix the acupuncture needle 320 into a proper position inside the needle case 340.

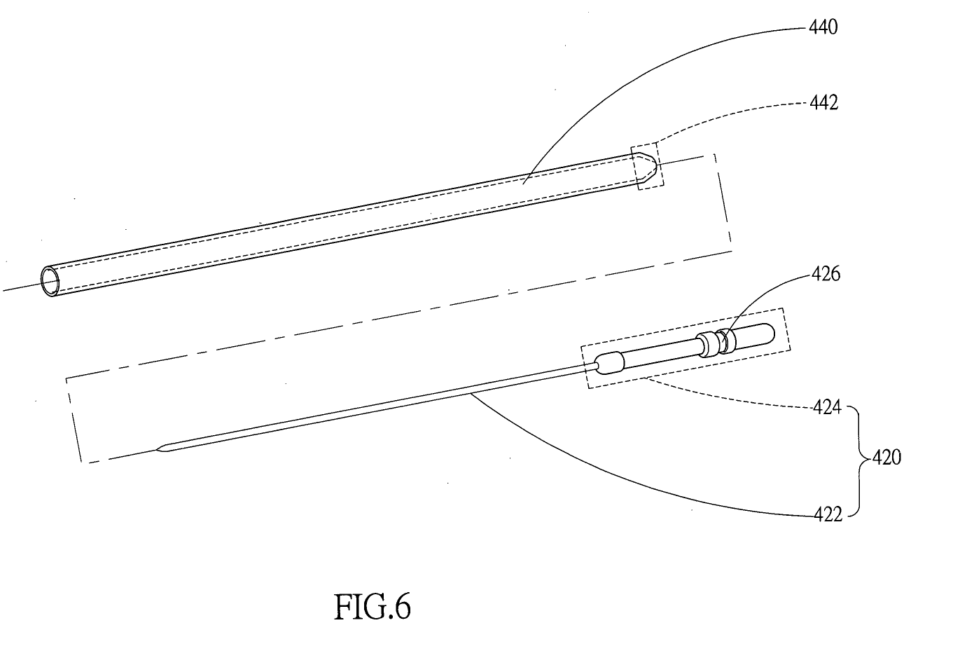

Still another preferred embodiment of the present invention is an improved acupuncture needle set. Referring to FIG. 6, the illustrated acupuncture needle set at least comprises an acupuncture needle 420 and a needle case 440. The mentioned needle case 440 may further comprise a first fastening structure 442. The first fastening structure is located at one opening of the needle case 440, and the mentioned opening has a smaller caliber than the caliber of another opening of the needle case 440.

The acupuncture needle 420 at least comprises a needle body 422 and a needle holder 424. The needle holder 424 could be made of plastic or other materials. The needle holder 424 may further comprise a second fastening structure 426. In accordance with this embodiment of the present invention, the second fastening structure 426 may further comprise a flute. The flute matched with the first fastening structure 442 in order to fix the acupuncture needle 420 to the proper position inside the needle case 440, as shown in the FIG. 6.

It should be appreciated and understood the design of the improved acupuncture needle set of this present invention, the detail of an embodiment of this invention would be explained as follows. Based on the design, the acupuncture needle could be placed inside the needle case at first. And it requires that the acupuncture needle is fixed to a proper position inside the needle case by matching the fastening structures respective disposed at the needle holder and the needle case. Before the insertion of the acupuncture needle, the whole acupuncture needle set is appropriately pinpointed to the desired acupoint by touching the lower end of the needle case with the skin of patient. Then appropriate pressure is punched by the acupuncturist on the needle holder to release the acupuncture needle from the fastening and insert the acupuncture needle into the skin of the patient. Finally, the needle case is removed and the acupuncture needle is twisted into a proper depth.

As is known, the acupuncture needle set in the prior art is usually manipulated by both of the acupuncturist's hands. During the insertion process, the needle case is handled on the desired acupoint by one hand, and the acupuncture needle is unfastened to release from the needle case by the other hand. In the consequence, it is not convenient for the acupuncturist. Moreover, since the manipulation of acupuncture needle set needs both hands, the acupuncturist may unconsciously or unintentionally shake the hand that handles the needle case when the other hand is releasing the acupuncture needle. Hence the acupuncture needle set in the prior art not only causes some inconvenience to the acupuncturist but also may reduce the accuracy of acupuncture.

In contrast of the acupuncture needle set of the prior art, the present invention provides an improved acupuncture needle set to be manipulated by single hand. Based on the design of this present invention, the needle case could be steadily handled in one of the acupuncturist's hands. At the same time, the acupuncture needle is punched by one finger (e.g. index finger) of the same hand. The resistant friction from these two fastening structures is overcome by the pressure of the finger and therefore the acupuncture needle is released. In short words, the insertion process based on this present invention can be performed by one hand of the acupuncturist. Therefore, the insertion process is more convenient and the inaccuracy induced from both hands manipulation in the prior art can also be reduced.

In summarized, the present invention disclosed an improved acupuncture needle set. The acupuncture needle set at least comprises an acupuncture needle and a needle case. The needle case further comprises a first fastening structure located at a proper position inside the needle case. The acupuncture needle at least comprises a needle body and a needle holder. Furthermore, the acupuncture needle set comprises a second fastening structure located at the needle holder. When the acupuncture needle is placed inside the needle case, the first fastening structure inside the needle case could be matched with the second fastening structure at the needle holder. The match prevents the acupuncture needle not to drop out of the needle case. It is also convenient to the acupuncturist to handle the whole acupuncture needle set. Moreover, only one hand is required to pinpoint the needle case at the desired acupoint, and appropriate pressure could be punched on the acupuncture needle by one finger of the same hand to overcome the resistant friction of these fastening structures. Therefore, the acupuncture needle would be released accurately on the desired acupoint. It is not only more convenient to the acupuncturist to perform acupuncture but also the inaccuracy induced from using both hands during acupuncture can be reduced.

The foregoing description is not intended to be exhaustive or to limit the invention to the precise forms disclosed. Obvious modifications or variations are possible in light of the above teachings. In this regard, the embodiment or embodiments discussed were chosen and described to provide the best illustration of the principles of the invention and its practical application to thereby enable one of ordinary skill in the art to utilize the invention in various embodiments and with various modifications as are suited to the particular use contemplated. All such modifications and variations are within the scope of the inventions as determined by the appended claims when interpreted in accordance with the breath to which they are fairly and legally entitled.

1. An apparatus for acupuncture needle set, comprising:
a needle case, wherein said needle case comprises a first fastening structure inside said needle case; and
an acupuncture needle, wherein said acupuncture needle comprises a needle body and a needle holder.

2. The apparatus of claim 1, further comprising a second fastening structure located at said needle holder.

3. The apparatus of claim 2, wherein the material of said second fastening structure includes plastic.

4. The apparatus of claim 1, wherein the material of said acupuncture needle includes metal.

5. The apparatus of claim 1, wherein said first fastening structure comprises a projecting point.

6. The apparatus of claim 1, wherein said first fastening structure comprises a circle of internal bulge inside said needle case.

7. The apparatus of claim 1, wherein said first fastening structure is at an opening of said needle case, and said opening has a smaller caliber than another opening of said needle case.

8. The apparatus of claim 2, wherein said second fastening structure comprises a flute.

9. An apparatus for acupuncture needle set, comprising:
a needle case, wherein said needle case comprises a first fastening structure inside said needle case; and
an acupuncture needle, wherein said acupuncture needle comprises a needle body, a needle holder, and a second fastening structure at said needle holder.

10. The apparatus of claim 9, wherein the material of said needle holder includes plastic.

11. The apparatus of claim 9, wherein said first fastening structure is at an opening of said needle case, and said opening has a smaller caliber than another opening of said needle case.

12. The set of claim 9, wherein said second fastening structure comprises a flute.
